(19) United States
(12) Patent Application Publication
(10) Pub. No.: US 2005/0148954 A1
(43) Pub. Date: Jul. 7, 2005

(54) Method for delivering a colonic lavage

(76) Inventor: Roy Abell, Duluth, GA (US)

Correspondence Address: Womble Carlyle Sandridge & Rice, PLLC, P.O. Box 7037, Atlanta, GA, 30357-0037, US

(21) Appl. No.: 10/960,822

(22) Filed: Oct. 7, 2004

Related U.S. Application Data
(60) Provisional application No. 60/509,743, filed on Oct. 8, 2003.

(51) Int. Cl. A61M 31/00

(52) U.S. Cl. 604/276; 600/187; 604/317

(57) ABSTRACT

Methods of delivering a colonic lavage with portable, lightweight bowel evacuation apparatus are provided. The methods generally include delivering a lavage liquid to the patient's colon and pulsating the lavage liquid by operating a manual pump.

### CROSS-REFERENCE TO RELATED APPLICATIONS

This application claims the benefit of the filing date of U.S. Provisional Patent Application, Ser. No. 60/509,743, filed on Oct. 8, 2003, and which is incorporated by reference as if fully set forth in its entirety herein.

### TECHNICAL FIELD

This invention generally relates to methods of delivering a colonic lavage.

### BACKGROUND

In the home and long-term care settings, if an individual is disabled, incapacitated and cannot mount a bedpan, bedside toilet chair or a bathroom toilet, the individual usually does not have an opportunity to evacuate on a regular basis because of their physical limitations including, but not limited to, nerve damaged bowels or constipating prescription drugs. This lack of opportunity can lead to serious buildup of toxic fecal material or fecal impactions that require medical attention. Traditionally, individuals in home care or long-term care facilities have been required to seek treatment for the fecal impactions in hospitals. Usually these hospital visits entail at least a three-day admission, barrages of orally-administered chemicals, series of ordinary enemas, and often rectal damaging digital removal of the stool using a gloved hand. If these procedures fail, then surgery has been the remaining alternative.

Traditional enemas, suppositories and laxatives also are employed frequently in home and long-term care facilities to facilitate proper bowel evacuation. Unfortunately, these traditional means often result in inadequate bowel evacuations and associated complications. Generally, conventional enemas provide for an infusion of water into the individual's colon, but do not provide a safe means for collecting or containing the wastewater and waste material within a closed system. Bowel management through enemas and other means may expose the nurse or attendant to contagious diseases. During the infusion step, the patient can experience cramping in their colon, necessitating stopping the procedure to allow the patient to evacuate. After receiving the infusion of water from an ordinary enema, the patient must quickly mount a bedpan, bedside toilet chair or bathroom toilet in order to dispose of the waste material. Not surprisingly, this step often results in bowel accidents that leave waste material on the bed or floor and, if the patient has a contagious disease, this further contributes to the danger of nurse/attendant exposure unnecessarily. As a result, many patients will elect to sit on the toilet while the ordinary enema is administered. However, this delivery position can be harmful to the patient, since the colon tends to be vertically aligned and the water pressure pressing sideways on the colon walls, over long-term practice, can cause the colon to dangerously enlarge permanently, which traditionally requires surgery to remedy.

Even after contending with these disadvantages, the patient's colon often is not completely cleaned because ordinary enemas and bowel evacuating chemicals usually cannot break up and remove fecal impactions or completely clean the colon in a reasonable amount of time, due in part to the fact that most conventional enemas include water reservoirs that can hold only about one liter of fluid. Accordingly, patients with limited mobility often can have bowel accidents after an enema or by other purging means, leaving them to lie or sit in their own fecal material. Medical studies have revealed that patients lying in their own stool for extended periods of time are susceptible to the occurrence of urinary tract infections, dermatological infections and decubitus ulcers, which often require hospital visits and surgery to treat.

While there are some alternatives to the conventional enema currently available, these conventional devices generally are burdened with various problems and shortcomings of their own. For example, many of the devices are permanently plumbed into the building, non-portable and suited for use only by medical personnel in the confines of a doctor's office, hospital, or other appropriate facility. Most are not suitable for personal use by a patient in his or her home, and certainly cannot be carried easily on trips away from home. In addition, most colonic devices administer large amounts of lavage liquid (typically 50 gal. of water) to the colon resulting in the removal of healthy blood electrolytes and ineffective removal of certain types of fecal impactions. Most of these devices include valves and specula that are formed of hard, uncomfortable components which are absent a means of securing the specula in the patient's rectum.

Consequently, there is a need for alternative methods for delivering a colonic lavage.

### SUMMARY

The present invention generally encompasses, among other things, methods for delivering a colonic lavage with pulsating action produced by operating a manual pump. The method generally entails delivering a lavage liquid to a patient's colon, pulsating the lavage liquid within the colon by operating a manual pump and then draining the liquid from the colon.

In one aspect, the present invention encompasses methods of administering a colonic lavage in which lavage liquid introduced into the colon is pulsed by operating a manual pump to provide desired agitation which tends to break up fecal matter and impactions. The method generally includes inserting the speculum into the rectum of the patient, delivering the lavage liquid through the speculum to the colon of the patient, pulsating the lavage liquid by operating a manual pump, and, draining the lavage liquid from the colon.

In another aspect, the method also includes delivering a medicant to the patient's colon through the speculum. The medicant can be pulsated by operating a manual pump and drained through the speculum from the patient's colon. A second charge of lavage liquid can be delivered to the patient's colon and pulsated for removing the medicants, if desired by the physician. The second charge of lavage liquid carrying the remains of medicants then can be drained from the colon through the speculum.

The method also can include inflating a cuff to retain the speculum in the patient's rectum and collecting waste material in a waste receptacle. The steps of the method can be carried out once or repeatedly to provide a cleansing colonic lavage.

In another aspect, the present invention encompasses methods for evacuating a patient's colon. The methods can include delivering a liquid to the patient's colon through the patient's rectum, pulsating the liquid by operating a manual pump, and draining the liquid into a sealed waste receptacle.

In yet another aspect, the present invention encompasses a method of delivering a colon lavage comprising providing a lavage liquid in a lavage liquid reservoir, and inserting a speculum into the colon of a patient. The method also includes delivering the lavage liquid through the speculum to the colon of the patient, and pulsating the lavage liquid in the colon of the patient by operating a manual pump. Draining the lavage liquid through the speculum to a sealed waste receptacle is also provided.

These and other aspects of the present invention are described below and shown in the drawings which are briefly described as follows.

### BRIEF DESCRIPTION OF THE DRAWINGS

FIG. 1 is a schematic illustration of a colonic lavage apparatus that can be used to carry out the methods of the present invention.

FIG. 2 is a perspective view of a speculum that can be used in the colonic lavage apparatus of FIG. 1.

FIG. 3 is a cross-sectional view of the speculum of FIG. 2.

FIG. 4 is a perspective view of another colonic lavage apparatus that can be used in carrying out the methods the present invention.

### DETAILED DESCRIPTION

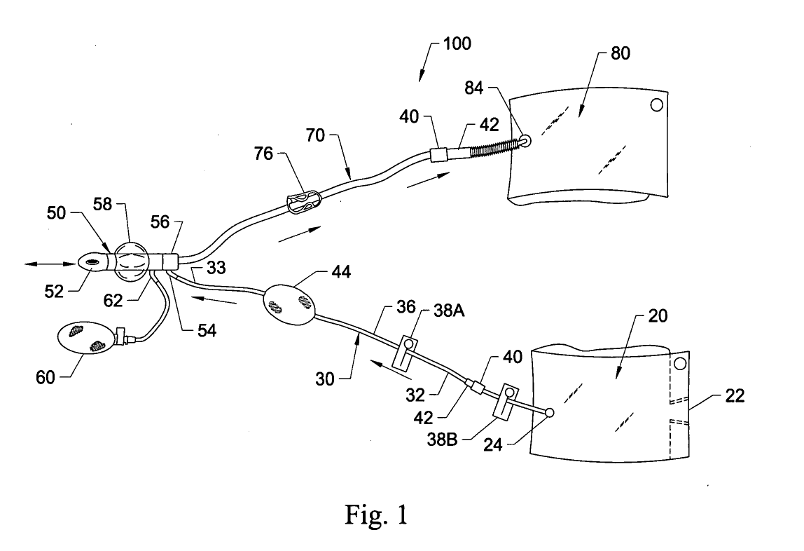

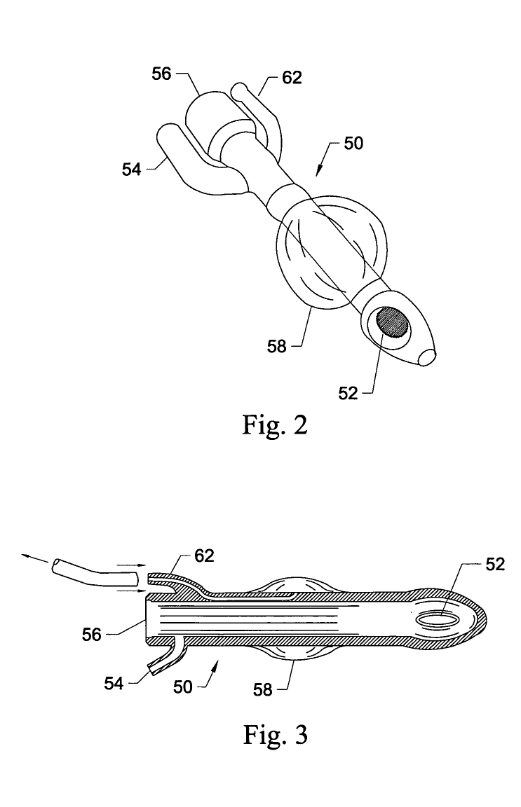

Referring now to the drawings, wherein like numerals refer to like features throughout the several views, FIGS. 1-4 illustrate features of an apparatus for delivering colonic lavage by methods of the present invention. As used herein, the term “colonic lavage” refers to the washing of the colon with one or more infusions of fluid into the colon. The term also encompasses the delivery to and/or removal from the colon of one or more medicants, alone or in conjunction with a washing. The delivery of medicants encompasses infusion of barium for X-ray procedures, chemotherapy compounds, and other medicinal compounds. While water typically is used as the lavage liquid in the colonic lavage, other liquids, medications, and materials can be delivered using the apparatus of the present invention.

FIG. 1 shows an apparatus 100 that can be used to deliver a colonic lavage according to the methods of the present invention. The apparatus 100 is designed to provide a colonic lavage with the capability of providing manually controlled pulsation to the lavage liquid or medicants through the use of a manual pump 44. As shown in FIG. 1, the manual pump 44 can be a squeeze bulb disposed in-line with a delivery line 30. The manual pump 44 can be formed of a rubber, polyvinyl elastomer or similar material. Since the apparatus 100 includes both a lavage liquid reservoir and a waste receptacle, it is not necessary to reposition the patient in order to remove fecal material from the colon. A colonic lavage or pulsing enema can be delivered to the patient while the patient is on their back or left side, thereby avoiding placing excessive pressure on the walls of the patient's colon.

The apparatus 100 comprises a speculum 50 that is configured and sized to be inserted into the patient's rectum. The speculum 50, which can be molded from a unitary piece of rubberized material such as silicone or polyvinyl elastomer, has a generally tubular body with a closed bulbous distal end in which a mouth 52 is formed. A pair of openings, an inlet 54 and an outlet 56, are formed in the proximal end of the speculum 50. A selectively inflatable cuff 58 is provided on the tubular body portion of the speculum 50 and is selectively inflated and deflated by use of a cuff pump 60.

The cuff pump 60 is in flow communication with an air inlet 62 on the speculum 50. The air inlet 62 ends under the inflatable cuff 58. The cuff pump 60 can be a polyvinyl squeeze bulb to which is attached an inline cutoff valve or with which a clamp attached to the tube leading to the air inlet 62 of the speculum 50. The tube leading from the cuff pump 60 to the air inlet 62 can be a flexible tubing made from vinyl or other polymeric material. One example of the tube has an inner diameter of about 3.0 mm and an outer diameter of about 4.3 mm and is about 30 cm in length. The inflatable cuff 58 includes one layer of silicone or vinyl tubing that has been folded over itself and glued at the joining ends for complete air enclosure to create a balloon. A small hole is created in the bottom layer of the balloon to allow air to enter the balloon from the air inlet 62 on the side of the speculum 50. An adhesive, disposed between the inflatable cuff 58 and the speculum 50, prevents air leakage.

A delivery line 30 is secured to inlet 54 of the speculum 50. The delivery line 30 is composed of a first section 32 and a second section 33. The sections of the delivery line 30 can be formed of vinyl or other polymeric tubing. In one embodiment, the first section 32 is made of vinyl tubing with an inner diameter of about 5.5 mm, an outer diameter of about 7.54 mm, and a length of about 91 cm; and the second section 33 is made of vinyl tubing with an inner diameter of about 9.5 mm, an outer diameter of about 12.7 mm and a length of about 61 cm. The manual pump 44 is disposed in-line with the delivery line 30 and connected to the second section 33. Male connector 42 and female connector 40 interconnect the delivery line 30 with lavage liquid reservoir 20. The first section 32 of the delivery line 30 is connected to a connector 24 on a lavage liquid reservoir 20, which is shown as a polymeric bag, but also can include other suitable containers and other available forms of connections between tube 30 and bag 20. A first valve 38 is operably connected to the delivery line 30 and allows liquid to flow from the lavage liquid reservoir 20 to the speculum 50. In FIG. 1 the first valve 38 is shown as a slide clamp connected to the first section 32 of the delivery line 30. However, alternative valve structures also are contemplated, such as a valve disposed in-line with the delivery line 30. For example the apparatus can be configured such that the manual pump operates a valve in line with the delivery line such that operating the manual pump opens and closes the valve, thereby pulsating the lavage liquid in the colon of the patient. These and other configurations of apparatus can be used to carry out the methods of the present invention.

The first valve 38A is disposed between the manual pump 44 and the end of the delivery line 30 to allow liquid delivered to the patient's colon through the speculum 50 to flow back to the manual pump 44, but not to the lavage liquid reservoir 20. The second valve 38B is disposed between connectors 40 and 42 and the lavage liquid reservoir 20 to facilitate closing the lavage liquid reservoir 20 during filling and/or connection to the delivery line 30. The lavage liquid reservoir 20 can be formed of a polymeric material, such as polyethylene, polypropylene, vinyl, polyvinyl chloride or rubber. In one embodiment, the lavage liquid reservoir is a single-use 3 mil polyethylene bag.

The lavage liquid reservoir 20 can have a handle, loop, eyelet or other means that can be used to hang the reservoir from a stand to elevate the reservoir above the patient and allow gravity feed of liquid to the patient. Typically, the lavage liquid reservoir 20 is placed approximately 2′ over a bed or other surface upon which the patient lies. The reservoir 20 can be configured to include a check valve that prevents the outflow of water through the fill opening when the reservoir 20 is being filled. This check valve can allow the reservoir 20 to be placed on a bed or other flat surface to allow the hand or other means of pressure to force the liquid from the reservoir 20 to the speculum 50. The first valve 38 controls the flow of liquid from the reservoir 20 to the mouth 54 of the speculum 50.

In an alternative aspect, another check valve, not shown, can be disposed in-line with the delivery line 30 or the manual pump 44. This check valve positioned on the reservoir side of the manual pump 44 also can be used to pump liquid from the lavage liquid reservoir 20 into the speculum 50, thereby providing an alternative to gravity feed. Once the desired amount of liquid is delivered to the patient's colon, the check valve 38 can be closed and the manual pump 44 operated as set forth below.

A drain line 70 is connected to the speculum 50 at outlet 56. The drain line 70 is in flow communication with the mouth 52 of the speculum 50, so as to allow liquids and material washed from the colon to be drained through the speculum. The drain line 70 also is in flow communication with a waste receptacle 80, which is shown in FIG. 1 as a polymeric bag, which can be a single use 3 mil polyethylene bag, connected to the end of the drain line 70 by connector 84. In one embodiment, the drain line 70 can be made of polyethylene or vinyl hose with an inner diameter of about 22 mm and a length of about 30.5 cm. Both the delivery line 30 and the drain line 70 can be constructed of collapsible polymeric material, such as polyethylene or polypropylene, so that the lines can be closed easily by valve clamps.

A second valve 76 is operably connected to the drain line 70 and regulates the flow of liquid between the speculum 50 and the waste receptacle 80. The second valve 76 also is shown as a slide clamp, but also can include alternative valve structures.

It will be understood that, with this configuration, when the second valve 76 is in its open configuration, waste liquid is free to flow through the outlet 56 of the speculum 50, through the drain line 70, and to the waste receptacle 80. Conversely, when the valve 76 is closed, the drain line 70 is closed and liquid/material cannot flow from the speculum 50 to the waste receptacle 80. Both the first and second valves 38 and 76 are manually operated for simplicity and to allow for the economical manufacture of the apparatus 100.

The construction and arrangement of the elements making up the assembly 100 are such that the speculum 50, delivery line 30, manual pump 44, lavage liquid reservoir 20, drain line 70 and waste receptacle 80 are disposable and can be removed or disconnected from the other elements of the system and replaced without disturbing or affecting the other elements of the system.

In practice, the lavage liquid reservoir 20 is filled with body temperature water or other suitable liquid through a valve, such as flap check valve 22 or opening formed therein. The reservoir 20 then is either suspended in an elevated position above the patient or placed on the bed or chair adjacent the patient. In one aspect, the lavage liquid reservoir 20 is designed to hold approximately 1 gallon of liquid, although other capacities are contemplated. The valves 38A and 38B are closed as the reservoir 20 is filled. After the lavage liquid reservoir 20 is filled, the valves 38A and 38B are kept closed. The lavage liquid reservoir 20 then is placed on the bed/surface, or hung from a hook/IV stand for gravity feed. The height of the lavage liquid reservoir 20 is such that the head or pressure level of the liquid is sufficient to deliver the liquid through the delivery line 30, manual pump 44 and speculum 50 into the patient's colon. As an alternative to gravity feed, the lavage liquid reservoir 20 can be placed on the bed or other surface and gently compressed with hand pressure to force the liquid through the apparatus and into the patient's colon. The female connector 40 then is connected to the male connector 42 attached to the manual pump 44.

The tip of the speculum 50 is lubricated with a water-soluble lubrication jelly and inserted into the rectum of the patient. The speculum 50 is inserted into the patient's rectum up to the collar or anal ring, and the cuff 58 is inflated to an extent sufficient to prevent the speculum 50 from accidentally slipping out of the rectum during the bowel evacuation procedure or material leakage during the procedure. The cuff pump 60 is repeatedly compressed to inflate the inflatable cuff 58 sufficiently to fill the rectal vault of the patient. Once inflated to the desired extent, the air line 62 is closed off with a suitable closure to maintain the cuff 58 in the inflated condition. The waste receptacle 80 is placed below the patient, such as on the floor, so that when the liquid is drained it can flow by gravity from the patient into the receptacle, thereby avoiding suction from being generated, which could possibly harm the patient.

The second valve 76 is closed to prevent liquid from draining from the speculum 50. The first valve 38 then is opened to allow liquid to flow from the reservoir 20 through the delivery line 30 into the speculum 50, through the mouth 52 and into the patient's colon. When the desired amount of lavage liquid is infused, the first valve 38 is closed.

With both the first and second valves 38 and 76 closed, either the patient or an assistant begins to compress the manual pump 44 to pressurize the liquid, thereby causing a pulsing action of liquid to enter the patient's colon and return to refill the squeeze bulb with liquid. Repeatedly squeezing or operating the manual pump 44 pulsates the liquid, which undulates in waves into and out of the patient's colon, thereby tending to break up the fecal material in the patient's colon. It has also been found through clinical trials that pulsating the lavage liquid triggers autonomic peristaltic action in the patient's colon, which further loosens, breaks up, and hydrates impacted waste material and helps move the fecal material towards the speculum 50 for evacuation through the speculum and into bag 80 when valve 76 is opened.

After pulsating the liquid, the valve 76 is opened, allowing the waste liquid and stool to exit the colon through the mouth 52 of the speculum 50 and drain into the drain line 70 and the waste receptacle 80. After the waste liquid drains into the waste receptacle 80, the process can be repeated. The process is repeated until it is determined that sufficient stool has been removed from the patient. Viewing the waste collected in the waste receptacle 80 is one way of determining the amount of stool that has been removed. The waste receptacle 80 can be detached from the rest of the apparatus 100 by disconnecting the male connector 42 from the female connector 40 and taken to a toilet for emptying. A handle or other convenient means can be provided to facilitate the transport of the waste receptacle 80 or for suspending it in a convenient location. The process generally provides for the repeated infusion of pulsing liquid into the patient's colon, leading to hydration and the breakup of the stool material into particles small enough to exit the colon through the speculum 50 and flow into the waste receptacle 80. The waste receptacle 80 can be sealed with the only opening formed therein connected to the drain line 70. In this configuration, the sealed waste receptacle 80 allows for fecal matter to be collected without exposing anyone to the biological material, thereby reducing the likelihood of disease transmission.

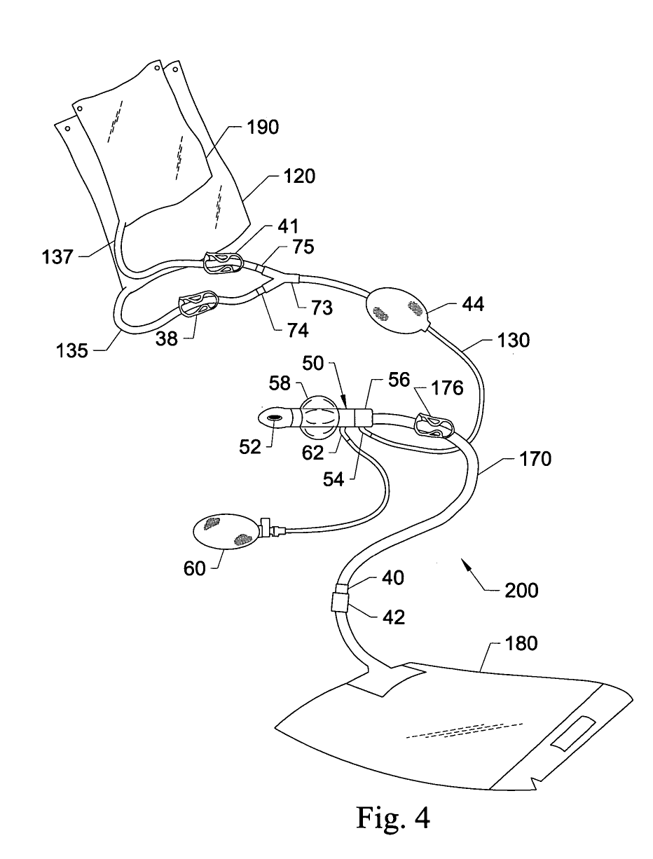

An alternative apparatus is shown in FIG. 4, the apparatus 200 includes a speculum 50 with a mouth 52 in fluid communication with a delivery line 130. A manual pump 44 is in-line with the delivery line 130. Unlike the delivery line 30, the delivery line 130 has a first branch 135 and a second branch 137. The first branch 135 is connected to a lavage liquid reservoir 120 and the second branch 137 is connected to a medicant reservoir 190. The first valve 38 regulates flow between the lavage liquid reservoir 120 and the speculum 50, whereas a medicant valve 41 regulates the flow between the medicant reservoir 190 and the speculum 50. The apparatus 200 also includes a waste receptacle 180 in flow communication with a drain line 170, which is in turn in flow communication with the mouth 52 of the speculum 50. A second valve 176 can open and close the drain line 170. As shown in FIG. 4, the first valve 38, the second valve 176 and the medicant valve 41 can be ratchet type pinch clamps, as well as other suitable valve structures.

The medicant reservoir 190 can hold one or more compounds or components for use in medical procedures, such as barium solutions for intestinal X-rays and chemotherapy mixtures as prescribed by a physician.

In use, the lavage liquid reservoir 120 is charged with a lavage liquid, such as water, and the medicant reservoir 190 is charged with a medicant, such as barium. Alternatively, prefilled reservoirs 120 or 190 can be connected to the first and second branches 135 and 137 of the delivery line 130. The speculum 50 is inserted and secured in the patient's rectum according to the steps described above. Initially the first valve 38, the medicant valve 41 and the second valve 176 are closed. Once the speculum 50 is inserted, the first valve 38 is opened to allow lavage liquid to flow from the lavage liquid reservoir 120 through the mouth 52 of the speculum 50 and into the patient's colon. The first valve 38 then is closed, and the manual pump 44 then is operated to pulse the lavage liquid in the colon, so as to wash, hydrate and/or break loose any material contained therein. The second valve 176 then is opened to allow the waste liquid to flow from the colon through the mouth 52 and the drain line 170 and into the waste receptacle 180. These steps can be repeated as necessary until the desired amount of washing and/or waste removal has been accomplished.

The second valve 176 is closed and the medicant valve 41 then is opened to allow the contents of the medicant reservoir 190 to flow through the delivery line 130 and the mouth 54 of the speculum 50 and into the colon. The medicant valve 41 can be closed and the manual pump 44 operated so as to pulsate the medicant material delivered to the colon. The second valve 176 then can be opened to allow the medicant material to be drained from the colon and into the waste receptacle 180.

The medicant material can be washed from the colon by redosing the second valve 176 and opening the first valve 38 to deliver a second charge of lavage liquid to the colon through the mouth 54 of the speculum 50. The second charge of lavage liquid can be pulsated as desired by operating the manual pump 44, after the first valve 38 has been reclosed. The waste liquid then can be drained as described above, thereby eliminating medicants, such as barium, from the patient after x-rays are made, which can be useful since barium has a tendency to harden and can cause life-threatening barium impactions.

The apparatus 100 and 200 can be employed in a variety of ways, such as in regular bowel maintenance programs and colon preparations for gastroenterology exams. Additionally, these apparatuses 100 and 200 can be used in emergency procedures, such as removing fecal impactions and emergency colonoscopies. The apparatuses 100 and 200 can be manufactured from light weight polymeric materials so as to allow for the easy transport and disposal of the components. Certain constructions of the apparatuses 100 and 200 are suitable for home use, thereby reducing and/or possibly eliminating the need for a patient to visit a physician or hospital for bowel evacuation.

The invention has been described herein in terms of particular embodiments and methodologies. It will be apparent to those of skill in the art, however, that various modifications might well be made to the illustrated embodiments within the scope of the invention. For example, while silicone or a silicone compound has been illustrated as one material from which the speculum is molded, other appropriate materials can be substituted. Further, the size and relative dimensions of the various components of the speculum, reservoirs and lines can be varied depending upon the intended use. For example, the manual pump or squeeze bulb can be differently shaped or sized providing a more pronounced pulsed action. Also, the manual pump can be positioned inline with the drain line of the assembly or in flow communication with the speculum through a line that is separated from both the delivery line and the drain line. Finally, the apparatus for delivering a colonic lavage and its various components as described herein may well be constructed with a wide variety of shapes and configurations that could differ from the illustrated examples and still be used to carry out the methods of the present invention. These and other additions, deletions, and modifications might well be made to the disclosed aspects by those of skill in the art without departing from the spirit and scope of the invention as set forth in the claims.

1. A method for delivering a colonic lavage comprising:
inserting a speculum into a rectum of a patient;
delivering a lavage liquid through a delivery line and the speculum to a colon of the patient;
pulsating the lavage liquid, wherein pulsating the lavage liquid comprises operating a manual pump; and,
draining the lavage liquid from the colon through a drain line.

2. The method of claim 1, further comprising delivering a medicant through the speculum to the colon of the patient.

3. The method of claim 2, further comprising pulsating the medicant in the colon of the patient, wherein pulsating the medicant comprises operating a manual pump.

4. The method of claim 2, further comprising draining the medicant through the speculum from the colon of the patient.

5. The method of claim 2, further comprising delivering a second charge of lavage liquid through the speculum to the colon of the patient.

6. The method of claim 5, further comprising draining the second charge of lavage liquid through the speculum from the colon of the patient.

7. The method of claim 1, further comprising closing a first valve to maintain the lavage liquid in the colon of the patient.

8. The method of claim 7, further comprising opening a second valve to drain the lavage liquid from the colon of the patient.

9. The method of claim 1, further comprising inflating a cuff to secure the speculum in the rectum of the patient.

10. The method of claim 1, further comprising draining the lavage liquid into a sealed waste receptacle in fluid communication with the speculum.

11. A method of evacuating a patient's colon comprising:
delivering a liquid to a patient's colon through the patient's rectum;
pulsating the lavage liquid, wherein pulsating the lavage liquid comprises operating a manual pump; and
draining the liquid from the patient's colon into a sealed waste receptacle.

12. The method of claim 11, further comprising delivering a medicant to the patient's colon through the patient's rectum.

13. The method of claim 12, further comprising pulsating the medicant in the patient's colon, wherein pulsating the medicant comprises operating a manual pump.

14. The method of claim 12, further comprising draining the medicant from the patient's colon into a sealed waste receptacle.

15. The method of claim 11, further comprising inserting a speculum into the patient's rectum and wherein the liquid is delivered to the patient's colon through the speculum.

16. The method of claim 15, further comprising inflating a cuff on the speculum.

17. The method of claim 12, further comprising delivering a second charge of lavage liquid to the colon of the patient.

18. The method of claim 17, further comprising draining the second charge of lavage liquid from the colon of the patient to the sealed waste receptacle.

19. A method of delivering a colon lavage comprising:
providing a lavage liquid in a lavage liquid reservoir,
inserting a speculum into the colon of a patient;
delivering the lavage liquid through the speculum to the colon of the patient;
pulsating the lavage liquid in the colon of the patient, wherein pulsating the lavage liquid comprises operating a manual pump;
draining the lavage liquid through the speculum to a sealed waste receptacle.

20. The method of claim 19, further comprising delivering a medicant through the speculum to the colon of the patient.

21. The method of claim 20, further comprising pulsating the medicant in the colon of the patient, wherein pulsating the medicant comprises operating a manual pump.

22. The method of claim 20, further comprising draining the medicant through the speculum from the colon of the patient to the sealed waste receptacle.

23. The method of claim 20, further comprising delivering a second charge of lavage liquid through the speculum to the colon of the patient.

24. The method of claim 23, further comprising draining the second charge of lavage liquid through the speculum from the colon of the patient to the sealed waste receptacle.

25. The method of claim 19, further comprising closing a first valve to maintain the lavage liquid in the colon of the patient.

26. The method of claim 25, further comprising opening a second valve to drain the lavage liquid from the colon of the patient to the sealed waste receptacle.

27. The method of claim 19, further comprising inflating a cuff to secure the speculum in the rectum of the patient.
